(19) United States
(12) Patent Application Publication
(10) Pub. No.: US 2025/0116591 A1
(43) Pub. Date: Apr. 10, 2025

(54) POROSITY AND/OR PERMEABILITY MEASUREMENT DEVICE AND METHOD

(71) Applicant: AALTO UNIVERSITY FOUNDATION SR, Aalto (FI)

(72) Inventors: Mikael MIETTINEN, Aalto (FI); Tuomas TIAINEN, Aalto (FI); Valtteri VAINIO, Aalto (FI); Raine VIITALA, Aalto (FI)

(73) Assignee: AALTO UNIVERSITY FOUNDATION SR, Aalto (FI)

(21) Appl. No.: 18/865,650

(22) PCT Filed: May 12, 2023
(86) PCT No.: PCT/FI2023/050265

§ 371 Date: Nov. 13, 2024

(30) Foreign Application Priority Data
May 13, 2022 (FI) 20227067

(51) Int. Cl. G01N 15/08; G01N 33/34

(52) U.S. Cl. CPC G01N 15/0826; G01N 33/346

(57) ABSTRACT

A device for measuring porosity and/or gas permeability of a sheet of material (9) comprises a measurement head comprising a body part (1) having a measurement face to be set towards the sheet of material, the body part comprising, on its measurement face, a first indentation defining a control volume (3) and a sealing arrangement (2, 5, 6, 7, 8) surrounding the first indentation to seal the control volume, and a control flow channel (11) forming a flow connection between the control volume and the outside of the body part for controlling the pressure in the control volume to create a pressure difference across the sheet of material to be measured. The sealing arrangement comprises a first aerostatic bearing for forming a contactless sealing between the body part (1) and the sheet of material (9) to be measured.

### TECHNICAL FIELD

The present specification relates to porosity and/or gas permeability measurement of sheet of materials such as paper, cardboard, or plastic.

### BACKGROUND OF THE INVENTION

Measurement of paperboard permeability is vital for quality control of the product. There are both on-line and off-line measurement methods that are used. Various off-line methods are outlined in the ISO 5636-3:2013 standard [1].

The methods are based on measuring the flow through a certain area of paper and have contacting seals that are pressed against the measured paper sheet [2,3]. In some methods, the sample is supported by a mesh or a porous surface [4,5].

Porosity and/or permeability measurements may be needed also, for example, for quality control of various plastic materials such as plastic films used for food packaging applications or as battery separator films. In the first case, the plastic sheets should not be porous or gas permeable, whereas in the latter example, controlled porosity and gas permeability of the films is important for the performance thereof.

One present solution describing prior art for permeability measurement has been disclosed in the U.S. Pat. No. 5,782,930. The invention is to measure air permeability of drying wire and consists of non-contact blow boxes. The solution disclosed in the said patent has the disadvantage that keeping the non-contact gap and seal the control volume simultaneously is difficult.

Another solution describing prior art for use of porosity is described in U.S. Pat. No. 4,676,091. This solution is intended for continuous measurement. Disadvantage of the method is that the measurement device is in contact with the measured sheet.

Further improved solutions are needed in the art, especially for on-line measurements.

### SUMMARY

This summary is provided to introduce a selection of concepts in a simplified form that are further described below in the detailed description. This summary is not intended to identify key features or essential features of the claimed subject matter, nor is it intended to be used to limit the scope of the claimed subject matter. The scope of protection sought for various embodiments of the present disclosure is set out by the independent claims.

In a first aspect, a device for measuring porosity and/or gas permeability of a sheet of material such as paper or cardboard may be implemented. The gas may be air or some other gas. The device comprises a measurement head comprising a body part having a measurement face configured to be set towards the sheet of material to be measured, the body part comprising, on its measurement face, a first indentation defining a control volume and a sealing arrangement surrounding the first indentation to seal the control volume, and a control flow channel forming a flow connection between the control volume and the outside of the body part for controlling the pressure in the control volume to create a pressure difference across the sheet of material to be measured.

“Porosity” may be considered as, for example, a measure of voids in the material. Such voids form empty volumes, “pores”, within the porous material. The pores can be interconnected, allowing passage of gas or fluids through the material. “Gas permeability” may be considered as a measure of the rate of gas transmission through such voids present in the material. A relationship between these measures can be derived by methods known in the art.

“A measurement head” may refer to any appropriate type of a measurement device, probe, setup, or arrangement configured to be positioned in such close proximity of the sheet of material to be measured that porosity and/or gas permeability measurements are possible.

“A measurement face” refers to the side of the body part to be positioned towards, i.e. facing, the sheet of material to be measured.

Sealing the control volume refers to the situation where the measurement head with the body part thereof is in use and brought into close proximity with a sheet of material to be measured such that the sheet of material delimits the control volume together with the body part first indentation. The sealing can also be considered being formed between the control volume and the ambient. The sealing thus closes the control volume in such a manner that the pressure in the control volume may be controlled.

The sealing arrangement “surrounding” refers to the sealing arrangement encircling at least partially, preferably substantially entirely, the first indentation, or at least its opening on the measurement face, and thus the control volume.

“A pressure difference across” refers to a pressure difference through the sheet of material, i.e. between the opposite sides of a specific location of the sheet of material.

Advantageously, the sealing arrangement comprises a first aerostatic bearing for forming a contactless sealing between the body part and the sheet of material to be measured. Contactless sealing may enable reliable, on-line measurement of, for example, moving sheets of material.

Aerostatic bearings as such are known in the art. An aerostatic bearing is basically based on a high-pressure gas-filled gap between two surfaces. Commonly, aerostatic bearings comprise of a bearing face that is one of the surfaces of the gap, and a means of delivering a controlled flow of gas into the bearing gap. In the present implementation, such bearing is used as a sealing between the body part and the sheet of material to be measured.

Any aerostatic bearing disclosed in this specification may comprise a sealing gas supply arrangement for supplying a sealing gas flow, and a restrictor mounted to the body part and configured to receive the sealing gas flow and transmit the received sealing gas flow through it for creating a sealing gap with an increased pressure between the restrictor and the sheet of material to be measured.

The sealing gas supply arrangement may comprise a sealing gas supply channel in a flow connection with a sealing gas distribution channel formed along an interface between the body part and the restrictor to distribute the sealing gas supply to the restrictor via the sealing gas distribution channel.

The sealing gas distribution channel may encircle substantially the entire perimeter of the first indentation.

“Substantially” refers here to that the sealing gas distribution channel may enclose entirely or at least close to entirely the first indentation and thus the control volume. In other words, it may be not necessary for sufficient sealing performance to have the sealing gas distribution channel to extend completely around the first indentation so as to for a closed loop channel.

Being formed along the interface between the body part and the restrictor refers to that the sealing gas distribution channel may be formed for example by means of a groove formed in the surface of the body part and/or the restrictor.

The sealing gas supply arrangement may comprise an inner sealing gas distribution channel and an outer sealing gas distribution channel, the inner sealing gas distribution channel being positioned between the control volume and the outer sealing gas distribution channel. In other embodiments, there may be even more sealing gas distribution channels. In embodiments with more than one sealing gas distribution channel, two or more of them may be in a flow connection with the same sealing gas supply channel. Alternatively, they may be separate sealing gas supply channels in flow connection with different sealing gas distribution channels.

Here “inner” thus refers to the side of the first indentation/control volume, and “outer” refers to the side of the ambient, i.e., the outside of the body part.

The restrictor may comprise, possibly being made of, a porous material such as porous graphite or of an initially solid material with small holes manufactured in it.

The first indentation may have an opening on the measurement face with an elongated shape. An elongated opening may be substantially straight or curved. An elongated opening may have substantially constant width. In other embodiments, the width may vary. An elongated opening may have a width in the range of about 6 to 10 mm, for example, about 8 mm, and a length of 4 to 6 times the width thereof. An elongated opening may be rectangular, possibly with rounded corners. In an embodiment, an elongated opening may have semi-circular or semi-elliptical ends.

i. The body part may comprise, on its measurement face, a second indentation surrounding the restrictor and defining an underpressure volume, and a second control flow channel forming a flow connection between the underpressure volume and the outside of the body part to create an underpressure in the underpressure volume for pulling, when in use, the sheet of material by the underpressure. In such embodiment, the body part may further comprise, on its measurement face, a second aerostatic bearing surrounding the second indentation for pushing, when in use, the sheet of material by an overpressure.

The device may comprise at least one pressure sensor and/or at least one flow sensor for measuring the gas flow and/or the pressure, respectively, in the control flow channel and/or in the control volume.

The device may comprise at least one pressure sensor and/or at least one flow sensor for measuring the sealing gas flow and/or the pressure in the sealing gas supply arrangement and/or in the sealing gap.

The measurement head may comprise two body parts arranged with their measurement faces facing each other for creating a pressure difference across a sheet of material to be measured positioned between the two body parts by means of different pressures arranged in the control volumes of the two body parts.

Using such two opposite body parts, the control volumes thereof may thus be positioned so as to lie at least partially aligned on opposite sides of the sheet of material to be measured.

The device may comprise a control arrangement, possibly comprising an electric control unit, configured to determine the porosity and/or gas permeability on the basis of pressure and/or flow in one or more of the following: the control flow channel(s), the control volume(s), the sealing gas supply arrangement(s), and the sealing gap(s).

Determination of the porosity and/or permeability may be based on calculations which may be carried out by means of analogous or digital electric means. It may also be possible to utilize mechanical and/or pneumatic means.

The control arrangement may comprise, possibly arranged in the electric control unit, at least one processor and at least one memory including computer program code, the at least one memory and the computer program code configured to, with the at least one processor, cause the device to carry out the determination of the porosity and/or gas permeability.

In a second aspect, a method for measuring porosity and/or gas permeability of a sheet of material (9) such as paper, cardboard, or plastic may be implemented. The method comprises arranging a sealed control volume adjacent to the sheet of material to be measured and controlling the pressure in the control volume to create a pressure difference across the sheet of material to be measured, characterized by using a first aerostatic bearing for forming a contactless sealing between the body part and the sheet of material to be measured.

The method may comprise arranging two sealed control volumes adjacent to the sheet of material to be measured, the two sealed control volumes lying at least partially aligned on opposite sides of the sheet of material to be measured, and controlling the pressures in the control volumes to create a pressure difference across the sheet of material to be measured.

The aerostatic bearing may comprise a sealing gas supply arrangement for supplying a sealing gas flow, and a restrictor mounted to the body part and configured to receive the sealing gas flow and transmit the received sealing gas flow through it for creating a sealing gap with an increased pressure between the restrictor and the sheet of material to be measured.

The method may comprise arranging an underpressure volume surrounding the first aerostatic bearing and controlling the pressure in the underpressure volume to create an underpressure therein for pulling the sheet of material by the underpressure. In such embodiment, the method may further comprise using a second aerostatic bearing surrounding the underpressure volume for pushing the sheet of material by an overpressure.

The method may comprise determining the porosity and/or gas permeability on the basis of pressure and/or flow in one or more of the following: the control flow channel(s), the control volume(s), the sealing gas supply arrangement(s), and the sealing gap(s).

Determination of the porosity and/or permeability may be based on calculations which may be carried out by means of analogous or digital electric means. It may also be possible to utilize mechanical and/or pneumatic means.

Determining the porosity and/or gas permeability may comprise compensating the effect of sealing gas leakage from a sealing gap (22) to a control volume (3, 20, 21) on the pressure and/or gas flow in the control volume.

The method may be implemented as an online measurement applied to a moving sheet of material, wherein determining the porosity and/or gas permeability comprises compensating the effect of gas conveyed to and/or out of a control volume in or on the moving sheet of material on the pressure and/or gas flow in the control volume.

In the implementation of the method, a device in accordance with any of the embodiments of the first aspect above may be utilized.

In the following description of examples and embodiments, terms may be used to refer to devices and elements thereof as well as to methods and steps thereof which differ from those term used above. It is however clear for the skilled person which terms used in the following description refer to the same devices, elements, methods, or steps as those discussed above.

Correspondence of some terms is listed below with the term used above presented first with a reference number, followed by corresponding term(s) used elsewhere in the description:
sheet of material to be measured 140, 240: sample
body part 111, 211, 311: chamber half; body;
control flow channel 115, 315: (inlet/exhaust) lines; connection; inlet connection;
control volume exhaust connection; control volume connection
control volume 114, 214a, 214b, 314: (inlet/exhaust) control volume; (inlet/exhaust) chamber;
aerostatic bearing 130: aerostatic seal; air bearing; seal;
sealing gas supply channel 131, 132: seal feed, seal supply connection
sealing gas distribution channel 133, 134: feed groove; supply groove
sealing gap 126, 226a, 226b: seal gap, air gap

Example embodiments are disclosed in the detailed description for porosity and/or gas permeability measuring systems, devices, and methods suitable to measure, for example, the porosity of a paper, cardboard, plastic, or other porous product which may be a bio-based product. In more generally, the measuring system, device, or method may be used to measure porosity and/or permeability of any sheet form material, i.e. a sheet of any (porous) material.

A measurement setup may comprise a measurement head with a single body part or a pair of opposite body parts implemented in the form of chamber halves that are positioned on both sides of the sample material.

A measurement setup may be instrumented with pressure and flow sensors in the chamber inlet and exhaust lines and in each seal feed. The individual sensors may allow to separate the seal leakage from the flow through the paper sheet.

The aerostatic seal leaks air or other gas used in the sealing into the inlet and exhaust chambers. Therefore, the flow through the sample can be calculated by subtracting the seal leakage flow from the exhaust flow. The seal leakage into the exhaust chamber can be measured from the inner feed groove of the seal. The flows at the exhaust chamber can be used as its pressure is closer to the ambient pressure than the high-pressure inlet chamber. The closer to ambient pressure results in cleaner division of the seal flow between the chamber and the ambient.

The flow through the sample material for air bearing was calculated by subtracting the seal leakage flow into the bottom chamber from the exhaust flow the bottom chamber.

### BRIEF DESCRIPTION OF THE DRAWINGS

The accompanying drawings, which are included to provide a further understanding of the invention and constitute a part of this specification, illustrate embodiments of the invention and together with the description help to explain the principles of the invention. In the drawings:

FIG. 1 presents a cross section of a measurement setup with a measurement head with a single body part and a sheet of material to be measured;

FIG. 2 presents a cross section of a measurement setup with a measurement head with two opposite body parts, and a sheet of material to be measured;

FIG. 3 presents a cross section of a body part of another measurement head;

FIG. 4 presents a top view of the body part of FIG. 5;

FIG. 5 presents an arrangement for gas supply and sensor system of a measurement head; and

FIG. 6 presents a flow chart of a method for measuring porosity and/or gas permeability.

The drawings of the Figures are schematic and drawn not to scale.

### DETAILED DESCRIPTION

Reference will now be made in detail to example embodiments, examples of which are illustrated in the accompanying drawings. The detailed description provided below in connection with the appended drawings is intended as a description of the present examples and is not intended to represent the only forms in which the present example may be constructed or utilized. The description sets forth the functions of the example and the sequence of steps or operations for constructing and operating the example. However, the same or equivalent functions and sequences may be accomplished by different examples.

An example embodiment comprises a method and device to measure the gas permeability of sheet-like materials. The measurement device comprises aerostatic bearing(s)/seal(s), control volume(s), and pressure and flow sensor(s).

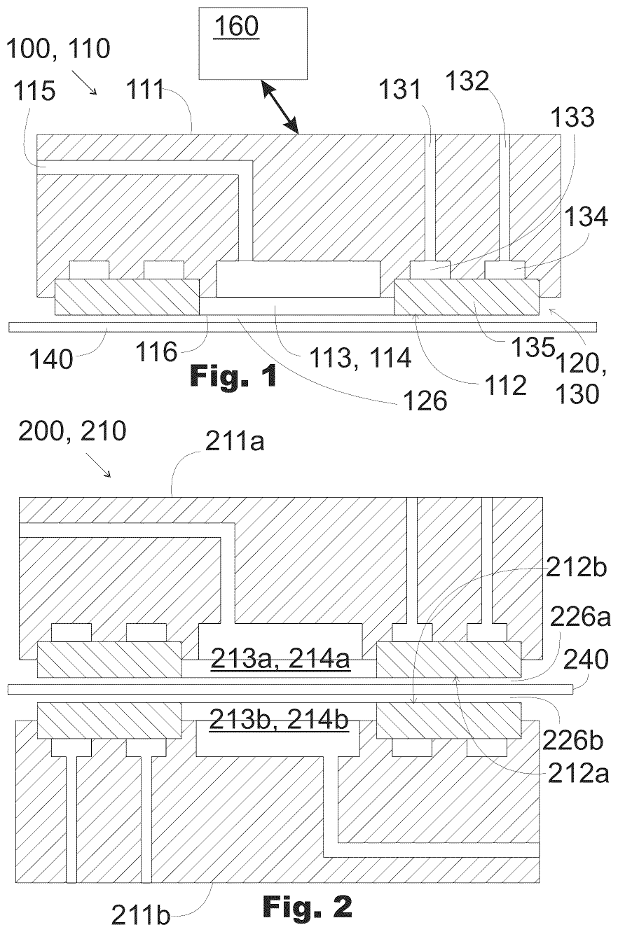

In the drawings of FIGS. 1 to 4, body parts representing measurement heads of devices are illustrated. In addition to the body parts illustrated in the drawings, the measurement heads as well as the devices may comprise any other appropriate further parts, elements, or components needed to implement a complete, operable measurement head or device.

FIG. 1 presents an example embodiment of the measurement device 100 used in a measurement setup. The device has a measurement head 110 with a single body part 111.

The body part 111 may be formed of, for example, a metal or plastic material. It has a measurement face 112 to be set towards the sheet of material 140 to be measured.

The body part comprises on its measurement face a first indentation 113 defining a control volume 114 and a sealing arrangement 120 surrounding the first indentation to seal the control volume. The indentation has an opening 116 on the measurement face 112. The opening defines the position, shape, and size of the indentation on the measurement face.

Outside the first indentation, the measurement face may be substantially planar.

There is a control flow channel 115 passing through the body part, forming a flow connection between the control volume 114 and the outside of the body part for controlling the pressure in the control volume. Thereby, a pressure difference may be created across the sheet of material to be measured.

In the single-body embodiment of FIG. 1, such pressure difference may be created by setting the pressure in the first control volume 114 below the ambient pressure. In the embodiment of FIG. 2 with two opposite body parts 211a, 211b, the two control volumes 214a, 214b can be used to create a pressure difference over the sheet of material 240 to be measured. The body parts of FIG. 2 may be in accordance with the body part of FIG. 1.

The sealing arrangement 120 comprises a first aerostatic bearing 130 for forming a contactless sealing between the body part 111 and the sheet of material 140 to be measured.

The first aerostatic bearing 130 comprises a sealing gas supply arrangement for supplying a sealing gas flow, a restrictor 135 mounted to the body part. The restrictor is configured to receive the sealing gas flow and transmit it through itself. Thereby, a sealing gap 126 with an increased pressure is created between the restrictor 135 and the sheet of material to be measured.

The sealing gas supply arrangement comprises an inner sealing gas supply channel 131 which is in a flow connection with an inner sealing gas distribution channel 133. The latter is formed so as to lie along an interface between the body part and the restrictor to distribute the sealing gas supply to the restrictor via the sealing gas distribution channel.

The sealing gas supply arrangement also comprises an outer sealing gas distribution channel 134 in a flow connection with an outer sealing gas supply channel 132. The inner sealing gas distribution channel is positioned between the control volume 114 and the outer sealing gas distribution channel 134.

In the example of FIG. 1, the inner and outer sealing gas distribution channels 133, 134 encircle substantially the entire perimeter of the first indentation 113 and the opening 116 thereof.

The opening 116 defining the first indentation may have any appropriate shape, such as a circular, an elliptical, or a rectangular shape. A rectangular, possibly elongated opening may have rounded ends.

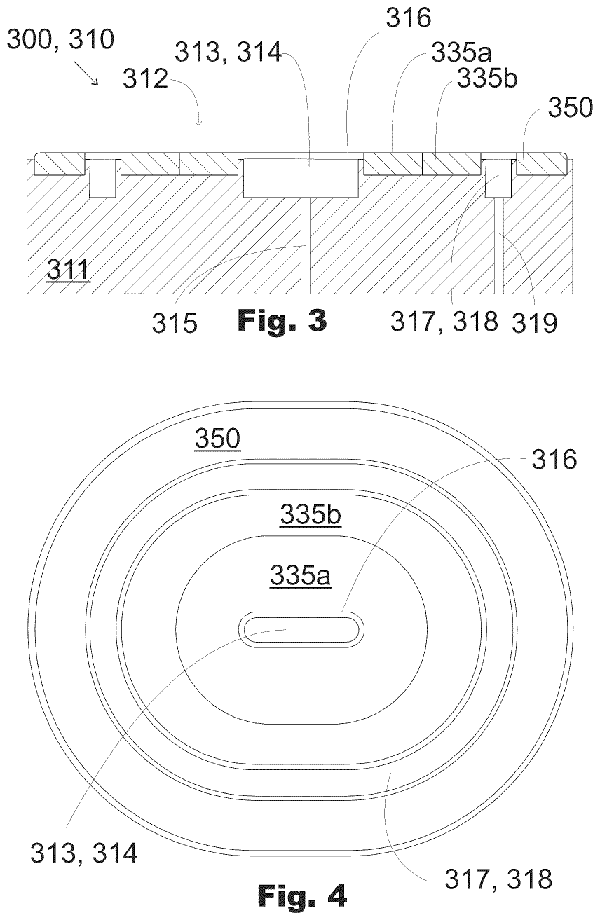

In the example of FIGS. 3 and 4, the opening 316 defining the first indentation 313 has a straight elongated shape with substantially semi-circular ends. It may have a width, for example, about 8 mm and a length about 30 mm.

An elongated shape with a limited width may advantageously facilitate stability of the sheet of material to be measured, whereby the limited width does not allow the sheet to be bent too much in the area of the pressurized control volume. In on-line measurements, the longitudinal direction of the opening may be set parallel to the direction of movement of the sheet to be measured.

In the example of FIGS. 3 and 4, the body part 311 of the measurement head 310 of the measurement device 300 comprises, on its measurement face 312, a second indentation 317 surrounding a two-part restrictor 335a, 335b. In other embodiments, a single-part restrictor may be used. The second indentation defines an underpressure volume 318, and a second control flow channel 319 forms a flow connection between the underpressure volume and the outside of the body part to create an underpressure in the underpressure volume. Thereby, the sheet of material to be measured may be pulled towards the body part by the underpressure. This may stabilize the position of the sheet of material, especially in the case of an on-line measurement.

The body part 311 comprises, on its measurement face 312, a second aerostatic bearing 350 surrounding the second indentation 317. Thereby, the sheet of material to be measured may be pushed, outside the underpressure volume, away from the body part by the increased pressure created by the bearing. This may further stabilize the position of the sheet of material, especially in the case of an on-line measurement.

In FIG. 3, sealing gas supply arrangement is not illustrated. It may be implemented in accordance with the examples of FIGS. 1 and 2. Corresponding gas supply arrangement may be implemented to supply and distribute gas to the second aerostatic bearing.

The devices of FIGS. 1 to 4 may comprise one or more pressure sensors and/or flow sensors in appropriate positions as defined in the summary sections.

As illustrated in FIG. 1, the device 100 comprises, as a part of a control arrangement, also a control unit 160 in a wired or wireless data transfer connection (illustrated by a two-directional arrow in FIG. 1) with the measurement head 110. The control unit is configured to determine the porosity and/or gas permeability on the basis of pressure and/or flow in one or more of the following: the control flow channel(s), the control volume(s), the sealing gas supply arrangement(s), and the sealing gap(s).

The measurement heads of FIGS. 1 to 4 comprise the body part and the aerostatic bearing/seal restrictor that is of annular shape. The restrictor and the body define the control volume. The control volume is connected to flow and pressure measurement sensors, and a means to generate a negative or positive pressure in the control volume. The seal is supplied with externally pressurized gas through inner seal supply connection and outer seal supply connection, which transmit pressurized gas through the inner supply groove and the outer supply groove. The supply grooves distribute the pressurized gas to the restrictor, that can be made of porous material such as graphite.

FIG. 1 presents an example embodiment of the permeability measurement device with a single measurement head or a single body part placed on one side of the measured sample. In this example, the control volume is brought to lower than ambient pressure on the other side of the sample by connecting the control volume to a low-pressure source, such as a ejector or vacuum pump by the control volume connection. The control volume is sealed between the body and the measured sample by the aerostatic seals. The restrictor of the seals is made of porous material, such as graphite. The restrictor can also be made with orifices, and use nozzles, pockets and grooves or any combination of those known in the art to distribute the seal gas to the seal gap. The restrictor (2) is used to transmit the high-pressure seal gas to the air gap to create a seal. The pressurized air is supplied to the restrictor material by one or multiple grooves. Here, the gas supply is trough inner seal supply groove that is connected to the inner seal supply connection and the outer seal supply groove that is connected to the outer seal supply connection. The seal supply has two distinct connections in order to measure the inner and outer gas flow separately, in order to measure the gas flow from the seal to the control volume.

FIG. 2 presents an example embodiment of permeability measurement device with two measurement heads or two body parts of a single measurement head, and two control volumes. The seals are arranged in a way that the measured sample s located in between the measurement heads or the two body parts. A pressure difference is created between the inlet control volume and the outlet control volume, by either increasing or reducing pressure in one or both control volumes. The inlet control volume is connected to gas supply through the inlet connection and the exhaust control volume is connected to exhaust or ambient through control volume exhaust connection The gas permeability of the measured sample can be obtained by measurement of control volume pressure or pressure difference, and by measuring the inlet and exhaust flows of the control volumes and by measuring the seal supply feeds, and calculating the amount of flow into the control volumes. The pressure and flow can be measured at single or multiple points, and the calculation of the gas permeability can use any of these measurements.

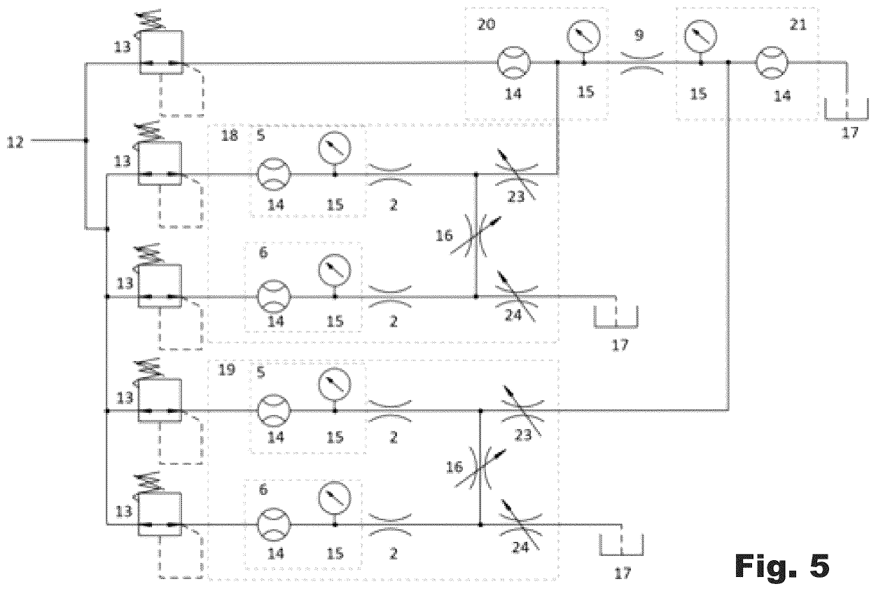

FIG. 5 presents an exemplary pneumatic system applied to porosity and permeability measuring device. Pressurized gas, often air is fed tot the system through point 12. Inlet pressure is regulated with pressure regulators 13 to all sections of the system. Structure 18 describes the inlet seal and structure 19 describes the exhaust seal. Gas feeding system is separated in such a way that both grooves 7 and 8 can be fed independently with two parallel sets of components 14, 15 and 2. Flow sensor 14 measures the air volume fed to feed channel. Pressure sensor 15 measures the pressure fed to each channel. Restrictor 2 describes the gas flow restriction accrued by the porous material. Adjustable restrictors 16, 23 and 24 presents the gas flow restriction accrued by the air gap. Restriction accrued by the air gap is adjustable because the height of the air gap is varying during the device is used. Restriction 23 describes the air flow restriction from the air gap to inlet 20 or outlet 21 control volume depending on which seal unit is observed. Restriction 24 describes the flow from the system to ambient. Restriction 16 describes the cross-sectional flow in the air gap which air has come from inner supply groove 7 and goes to ambient 17 or air which has come to air gap from outer supply groove 8 and goes to control volume 20 or 21. Inlet control volume 20 is pressurized and the flow to the control volume is measured with flow sensor 14 and the pressure in the inlet control volume is measured with pressure sensor 15. Restriction 9 presents the sample between inlet seal 18 and outlet seal 19. Pressure in the outlet control volume 21 is measured with pressure sensor 15 and the flow from the outlet control volume to ambient 17 is measured with flow sensor 21.

The seal supply can be arranged with one or multiple supply grooves, and they can be connected to flow and pressure sensors and regulators individually or together as a single group or multiple groups. The flow to the control volume can be interpreted from the measurement of any of the flows to the seal and from any of the flows to one or multiple control volume connections.

Further features of the method directly result for example from the functionalities and parameters of the deflection compensated roll as described in the appended claims and throughout the specification and are therefore not repeated here. Different variations of the methods may be also applied, as described in connection with the various example embodiments.

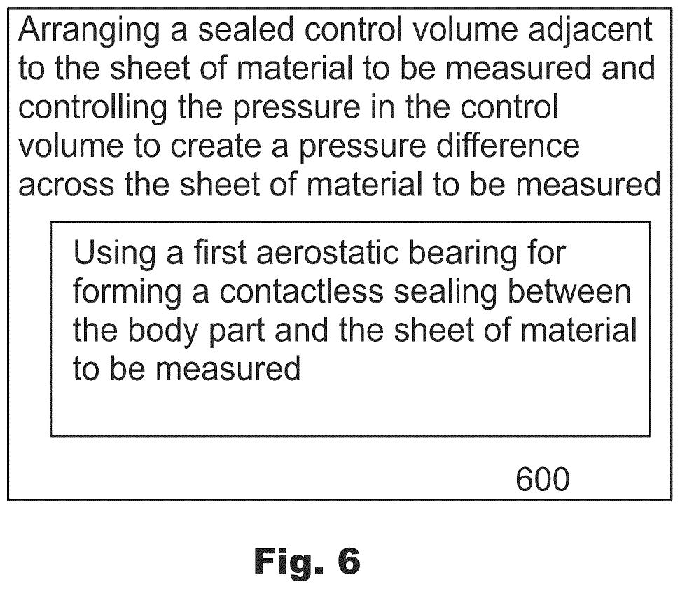

The method 600 of FIG. 6 for measuring porosity and/or gas permeability may be carried out by any of the devices discussed above with reference to FIGS. 1 to 5.

The method comprises arranging a sealed control volume adjacent to the sheet of material to be measured and controlling the pressure in the control volume to create a pressure difference across the sheet of material to be measured. A first aerostatic bearing is used for forming a contactless sealing between the body part and the sheet of material to be measured.

The permeability measurement device may be configured to perform or cause performance of any aspect of the methods described herein.

Any range or value given herein may be extended or altered without losing the effect sought. Also, any embodiment may be combined with another embodiment unless explicitly disallowed.

Although the subject matter has been described in language specific to structural features and/or acts, it is to be understood that the subject matter defined in the appended claims is not necessarily limited to the specific features or acts described above. Rather, the specific features and acts described above are disclosed as examples of implementing the claims and other equivalent features and acts are intended to be within the scope of the claims.

It will be understood that the benefits and advantages described above may relate to one embodiment or may relate to several embodiments. The embodiments are not limited to those that solve any or all of the stated problems or those that have any or all of the stated benefits and advantages. It will further be understood that reference to ‘an’ item may refer to one or more of those items.

The steps or operations of the methods described herein may be carried out in any suitable order, or simultaneously where appropriate. Additionally, individual blocks may be deleted from any of the methods without departing from the scope of the subject matter described herein. Aspects of any of the embodiments described above may be combined with aspects of any of the other embodiments described to form further embodiments without losing the effect sought.

The term ‘comprising’ is used herein to mean including the method, blocks, or elements identified, but that such blocks or elements do not comprise an exclusive list and a method or apparatus may contain additional blocks or elements.

It will be understood that the above description is given by way of example only and that various modifications may be made by those skilled in the art. The above specification, examples and data provide a complete description of the structure and use of exemplary embodiments. Although various embodiments have been described above with a certain degree of particularity, or with reference to one or more individual embodiments, those skilled in the art could make numerous alterations to the disclosed embodiments without departing from scope of this specification.

1-24. (canceled)

25. A device for measuring porosity and/or gas permeability of a sheet of material such as paper or cardboard, the device comprising a measurement head comprising a body part having a measurement face configured to be set towards the sheet of material to be measured, the body part comprising, on its measurement face, a first indentation defining a control volume and a sealing arrangement surrounding the first indentation to seal the control volume, and a control flow channel forming a flow connection between the control volume and the outside of the body part for controlling the pressure in the control volume to create a pressure difference across the sheet of material to be measured, characterized in that the sealing arrangement comprises a first aerostatic bearing for forming a contactless sealing between the body part and the sheet of material to be measured.

26. A device as defined in claim 25, wherein the first aerostatic bearing comprises a sealing gas supply arrangement for supplying a sealing gas flow, and a restrictor mounted to the body part and configured to receive the sealing gas flow and transmit the received sealing gas flow through it for creating a sealing gap with an increased pressure between the restrictor and the sheet of material to be measured.

27. A device as defined in claim 26, wherein the sealing gas supply arrangement comprises a sealing gas supply channel in a flow connection with a sealing gas distribution channel formed along an interface between the body part and the restrictor to distribute the sealing gas supply to the restrictor via the sealing gas distribution channel.

28. A device as defined in claim 27, wherein the sealing gas distribution channel encircles substantially the entire perimeter of the first indentation.

29. A device as defined in claim 27, wherein the sealing gas supply arrangement comprises an inner sealing gas distribution channel and an outer sealing gas distribution channel, the inner sealing gas distribution channel being positioned between the control volume and the outer sealing gas distribution channel.

30. A device as defined in claim 26, wherein the restrictor comprises porous material such as porous graphite.

31. A device as defined in claim 26, wherein the first indentation has an opening on the measurement face with an elongated shape.

32. A device as defined in claim 31, wherein the opening has a width in the range of about 6 to 10 mm, and a length of 4 to 6 times the width thereof.

33. A device as defined in claim 26, wherein the body part comprises, on its measurement face, a second indentation surrounding the restrictor and defining an underpressure volume, and a second control flow channel forming a flow connection between the underpressure volume and the outside of the body part to create an underpressure in the underpressure volume for pulling, when in use, the sheet of material by the underpressure.

34. A device as defined in claim 33, wherein the body part comprises, on its measurement face, a second aerostatic bearing surrounding the second indentation for pushing, when in use, the sheet of material by an overpressure.

35. A device as defined in claim 26, further comprising at least one pressure sensor and/or at least one flow sensor for measuring the sealing gas flow and/or the pressure in the sealing gas supply arrangement and/or in the sealing gap.

36. A device as defined in claim 26, further comprising a control arrangement, possibly comprising an electric control unit, configured to determine the porosity and/or gas permeability on the basis of pressure and/or flow in one or more of the following: the control flow channel(s), the control volume(s), the sealing gas supply arrangement(s), and the sealing gap(s).

37. A device as defined in claim 36, wherein the control arrangement comprises, possibly arranged in the electric control unit, at least one processor and at least one memory including computer program code, the at least one memory and the computer program code configured to, with the at least one processor, cause the device to carry out the determination of the porosity and/or gas permeability.

38. A device as defined in claim 25, further comprising at least one pressure sensor and/or at least one flow sensor for measuring the gas flow and/or the pressure, respectively, in the first control flow channel and/or in the control volume.

39. A device as defined in claim 25, wherein the measurement head comprises two body parts arranged with their measurement faces facing each other for creating a pressure difference across a sheet of material to be measured positioned between the two body parts by means of different pressures arranged in the control volumes of the two body parts.

40. A method for measuring porosity and/or gas permeability of a sheet of material such as paper, cardboard, or plastic, the method comprising arranging a sealed control volume adjacent to the sheet of material to be measured and controlling the pressure in the control volume to create a pressure difference across the sheet of material to be measured, characterized by using a first aerostatic bearing for forming a contactless sealing between the body part and the sheet of material to be measured.

41. A method as defined in claim 40, comprising arranging two sealed control volumes adjacent to the sheet of material to be measured, the two sealed control volumes lying at least partially aligned on opposite sides of the sheet of material to be measured, and controlling the pressures in the control volumes to create a pressure difference across the sheet of material to be measured.

42. A method as defined in claim 40, wherein the aerostatic bearing comprises a sealing gas supply arrangement for supplying a sealing gas flow, and a restrictor mounted to the body part and configured to receive the sealing gas flow and transmit the received sealing gas flow through it for creating a sealing gap with an increased pressure between the restrictor and the sheet of material to be measured.

43. A method as defined in claim 42, comprising determining the porosity and/or gas permeability on the basis of pressure and/or flow in one or more of the following: the control flow channel(s), the control volume(s), the sealing gas supply arrangement(s), and the sealing gap(s).

44. A method as defined in claim 43, wherein determining the porosity and/or gas permeability comprises compensating the effect of sealing gas leakage from a sealing gap to a control volume on the pressure and/or gas flow in the control volume.

45. A method as defined in claim 40, comprising arranging an underpressure volume surrounding the first aerostatic bearing and controlling the pressure in the underpressure volume to create an underpressure therein for pulling the sheet of material by the underpressure.

46. A method as defined in claim 45, comprising using a second aerostatic bearing surrounding the underpressure volume for pushing the sheet of material by an overpressure.

47. A method as defined in claim 40, implemented as an online measurement applied to a moving sheet of material, wherein determining the porosity and/or gas permeability comprises compensating the effect of gas conveyed to and/or out of a control volume in or on the moving sheet of material on the pressure and/or gas flow in the control volume.
